(19) United States
(12) Patent Application Publication
(10) Pub. No.: US 2016/0367677 A1
(43) Pub. Date: Dec. 22, 2016

(54) LIPID NANODISCS AND NANORODS AS MODULATORS OF CLOTTING FACTOR FUNCTION IN VIVO

(71) Applicant: Svetla Stoilova-McPhie, Dallas, TX (US)

(72) Inventor: Svetla Stoilova-McPhie, Dallas, TX (US)

(73) Assignee: Board of Regents, The University of Texas System, Austin, TX (US)

(21) Appl. No.: 15/186,261

(22) Filed: Jun. 17, 2016

Related U.S. Application Data
(60) Provisional application No. 62/182,210, filed on Jun. 19, 2015.

(51) Int. Cl. A61K 47/24; A61K 9/51; G01N 33/49; A61K 38/37

(52) U.S. Cl. CPC A61K 47/24; A61K 38/37; A61K 9/5123; G01N 33/49

(57) ABSTRACT

The present invention includes composition and methods of using a lipid nanodisk or nanotube composition comprising a lipid composition of phosphatidylserine and galactosylceramide and a membrane-bound Factor VIII protein, a membrane-bound Factor IX protein, a membrane-bound Factor VIII-Factor IX protein complex, or a membrane-bound Factor V-Factor X protein complex in or about the lipid nanodisks or nanotubes.

### CROSS-REFERENCE TO RELATED APPLICATIONS

This application claims priority to U.S. Provisional Application Ser. No. 62/182,210, filed Jun. 19, 2015, the entire contents of which are incorporated herein by reference.

### STATEMENT OF FEDERALLY FUNDED RESEARCH

None.

### TECHNICAL FIELD OF THE INVENTION

The present invention relates in general to the field of blood clotting, and more particularly, to novel lipid nanodiscs and nanorods as modulators of clotting using, e.g., Factor VIII, Factor IX protein, a Factor VIII-Factor IX protein complex, or a Factor V-Factor X protein complex in vivo.

### BACKGROUND OF THE INVENTION

Without limiting the scope of the invention, its background is described in connection with blood clotting.

Factor VIII in its active form serves as the co-factor to the serine protease Factor IXa within the membrane-bound intrinsic tenase complex. The assembly of the FVIIIa-FIXa complex on the activated platelet surface increases FIXa proteolytic activity and Factor Xa generation more than 100,000 times, which is critical for normal hemostasis (1, 2). FVIII is a heterodimer composed of a variable length heavy chain (HC: A1-A2-B) of 90-200 kDa containing variable length B-domain and a constant length light chain (LC: A3-C1-C2) of 80 kDa. The LC and HC are non-covalently linked via divalent Ca²⁺ ion(s) (1). Factor VIII is activated by Thrombin resulting in the cleavage of the entire B domain and separation of the A2 and A1 domains (3). Activated FVIII (FVIIIa) is a heterotrimer composed of non-covalently linked A1, A2 domains and the LC. The A1 and LC retain the metal ion-dependent linkage through the A1-A3 domains, whereas the association of A2 to A1 is mediated solely by hydrophobic and electrostatic interactions (1, 2, 4-10). The A2 and A3 domains contain the main protease (FIXa) binding sites and the C domains hold the main membrane binding sites (11-13). The FVIIIa binds to the FIXa with high affinity in a membrane-dependent manner resulting in the intrinsic tenase (FVIIIa-FIXa) complex assembly on the activated platelet surface during the propagation phase of critical for sufficient Thrombin generation (1, 14, 15).

Defect or deficiency of FVIII is cause for Hemophilia A, and Hemophilia A is effectively corrected by repetitive intravenous injection of recombinant or plasma-derived FVIII concentrates to prevent recurring bleeding (16). Despite the critical role of FVIII for normal hemostasis the knowledge of its membrane-bound organization alone or within the intrinsic tenase complex is incomplete, due to the complexity of its domain organization and instability of the active form—FVIIIa. This lack of structural information hampers drug discovery that can effectively regulate the activity of the intrinsic tenase complex and improve the design of new pro- and anti-coagulant drugs.

In the last decade a new approach for functional and structural studies of membrane-associated proteins was developed based on lipid nanodiscs (ND) technologies that can mimic the lipid composition of physiological membranes. Lipid ND have been previously employed for functional assays of coagulation factors and complexes, such as the Tissue Factor-Factor VIIa complexes and showed the advantage of this approach over phospholipid vesicles and artificial membrane bilayers in terms of precise control of the local membrane composition that surrounds the coagulation complexes (17). ND are small, ˜10 nm in diameter size circular lipid bilayer membrane patches stabilized by two molecules of amphiphilic membrane scaffolding proteins (MSP). They are produced by a self-assembly process, which creates a preparation of homogenous and highly stable lipid bilayer discs with controlled lipid composition. Such small size of the available membrane surface can allow the assembly macromolecular complexes that contain the protein molecules of interest attached to the ND membrane with a size and macromolecular mass suitable for visualization and structure determination by single particle EM (18). The geometry (flat membrane discs) and homogenous distribution of the ND makes them more desirable for structural studies than liposomes, which are more heterogeneous in size with a higher curvature and larger membrane surface that leads to overlap membrane-bound molecules in the EM images (19).

### SUMMARY OF THE INVENTION

In another embodiment, the present invention includes a lipid nanodisk or nanotube composition comprising: a lipid composition comprising phosphatidylserine and galactosylceramide and a membrane-bound Factor VIII protein, a membrane-bound Factor IX protein, a membrane-bound Factor VIII-Factor IX protein complex, or a membrane-bound Factor V and Factor X proteins and their complex in or about the lipid nanodisks or nanotubes. In one aspect, the membrane-bound Factor VIII is a full length or B-domain deleted variant of recombinant FVIII. In another aspect, the phosphatidylserine and galactosylceramide lipids and any derived lipids are at a ratio from 20 to 80 percent by volume. In another aspect, the phosphatidylserine is used as a 20 to 50%, or 50 to 80% liquid composition. In another aspect, the membrane-bound Factor VIII to lipid ratio is from 1:47 to 1:72. In another aspect, the membrane-bound Factor VIII to lipid ratio is from 1:40 to 1:150. In another aspect, the nanodisk is in a phosphatidylserine 80% lipid composition and 1:47 membrane-bound Factor VIII to lipid ratio. In another aspect, the composition further comprises one or more amphiphilic membrane scaffolding proteins (MSP) and/or their derivatives of Apolipoproteins I. In another aspect, the Factor VIII protein, the Factor IX protein, the Factor VIII-Factor IX protein complex, or the Factor V, Factor X proteins and their complex is an activated form of the protein. In another aspect, the Factor VIII is activated truncated protein that forms a functional intrinsic tenase complex with a human FIXa on a negatively charged phospholipid surface.

Another embodiment of the present invention includes a method of making a lipid nanodisk or nanotube comprising: dissolving phosphatidylserine and galactosylceramide lipids in an organic solvent; evaporating the organic solvent under a noble gas, reconstituting the lipids in an aqueous buffered solution with Na cholate, warming and sonicating the lipids until they are in solution, adding a membrane-bound Factor VIII protein, a membrane-bound Factor IX protein, a membrane-bound Factor VIII-Factor IX protein complex, or a membrane-bound Factor V and Factor X proteins and their complex into the lipids, and removing the Na Cholate with beads, wherein the NaCholate-beads that are removed by centrifugation. In one aspect, the membrane-bound Factor VIII is a full-length or a B-domain deleted variant of recombinant FVIII. In another aspect, the phosphatidylserine and galactosylceramide lipids and any derived lipids are at a ratio from 20 to 80 percent by volume. In another aspect, the phosphatidylserine is used as a 20 to 50%, or 50 to 80% liquid composition. In another aspect, the membrane-bound Factor VIII to lipid ratio is from 1:47 to 1:72. In another aspect, the membrane-bound Factor VIII to lipid ratio is from 1:40 to 1:150. In another aspect, the nanodisk is in a phosphatidylserine 80% lipid composition and 1:47 membrane-bound Factor VIII to lipid ratio. In another aspect, the composition further comprises one or more amphiphilic membrane scaffolding proteins (MSP) and/or their derivatives of Apolipoproteins I. In another aspect, the Factor VIII protein, the Factor IX protein, the Factor VIII-Factor IX protein complex, or the Factor V, Factor X proteins and their complex is an activated form of the protein. In another aspect, the Factor VIII is activated truncated protein that forms functional an intrinsic tenase complex with a human FIXa on a negatively charged phospholipid surface.

Yet another embodiment includes a method of treating a disease of blood coagulation comprising: identifying a subject in need of treatment for the disease of blood coagulation caused by a mutation in at least one of Factor V, Factor VIII, Factor IX, or Factor X; and providing the subject with a therapeutically effective amount of a phosphatidylserine and galactosylceramide nanodisk or nanotube composition comprising a membrane-bound Factor VIII protein, a membrane-bound Factor IX protein, a membrane-bound Factor VIII-Factor IX protein complex, or a membrane-bound Factor V and Factor X proteins complex, wherein the Factor V, Factor VIII, Factor IX, or Factor X are provided in an active or inactive form.

Yet another embodiment includes a method of determining the effectiveness of a candidate drug that impacts Factor V, VIII, IX and/or X activity, the method comprising: (a) obtaining a serum or plasma from a normal subject and a subject with an abnormality in blood clotting; (b) preparing a stable lipid nanodisk or nanotube comprising phosphatidylserine and galactosylceramide that comprises at least one of: a membrane-bound Factor VIII protein, a membrane-bound Factor IX protein, a membrane-bound Factor VIII-Factor IX protein complex, or a membrane-bound Factor V-Factor X protein complex; (c) combining the serum or plasma from the normal and from the abnormal subjects; and (d) imaging the membrane-bound Factor VIII protein, the membrane-bound Factor IX protein, the membrane-bound FactorVIII-Factor IX protein, or the membrane-bound Factor VIII-Factor IX protein complex in the normal and the abnormal serum by at least one of electron microscopy or single-particle analysis; (e) adding the candidate drug to the nanodisks or nanotubes, and (f) imaging the nanodisks or nanotubes in the normal and the abnormal serum by at least one of electron microscopy or single-particle analysis to determine the structural differences in nanodisks or nanotubes comprising the membrane-bound Factor VIII protein, the membrane-bound Factor IX protein, the membrane-bound FactorVIII-Factor IX protein, or the membrane-bound Factor VIII-Factor IX protein complex in the presence or absence of the candidate drug.

The present invention also includes compositions and methods for making a stable Factor VIII-Nanodisk (FVIII-ND) complexes suitable for structural studies by EM and single-particle analysis. The FVIII-ND of the present invention is a B-domain deleted recombinant porcine FVIII, which has a 84% sequence identity with the human FVIII analogue and is used as a drug for Hemophilia A in patients who develop antibodies against human FVIII (24, 25). Recombinant porcine FVIII lacking the B-domain has much higher expression level in cell culture than the human FVIII analogue, has a higher stability in its activated form and forms functional intrinsic tenase complexes with human FIXa on negatively charged phospholipid surface, which makes it an ideal candidate for structural and functional studies by EM at close to physiological conditions (26-28). To achieve a homogenous population of functional FVIII molecules bound to the PS-rich ND, the ND were first assembled at different PS concentration and MSP1D1 to lipid ratio. The ND population that was the most amenable for single particle analysis of the negatively stained FVIII-ND complexes adsorbed on amorphous carbon film was selected. The calculated FVIII membrane-bound organization at 25 Å resolution showed that FVIII organizes preferentially as a dimer at the ND surface on both or one side of the ND. This organization confirms the inventor's previous analysis for human and porcine FVIII helically organized on lipid nanotubes (LNT) with the same lipid composition (29, 30). The developed algorithm for monitoring the FVIII membrane-bound organization, as bound to ND is a significant step towards resolving the FVIIIa-FIXa functional organization on the activated platelet membrane. The lipid nanotechnologies employed in this study were also tested for their stability to stabilize active coagulation complexes in vivo.

### BRIEF DESCRIPTION OF THE DRAWINGS

For a more complete understanding of the features and advantages of the present invention, reference is now made to the detailed description of the invention along with the accompanying figures and in which:

FIGS. 1A to 1E are graphs that summarize the data obtained from electron microscope photographs of nanodisks (ND) at different lipid composition. Electron micrographs of negatively stained GC-based ND containing the following ratios of phosphatidylserine (PS) and galactosylceramide and a membrane scaffolding protein (MSP), in this case membrane scaffolding protein 1D1 (MSP1D1) (Sigma-Aldrich), are summarized in the following graphs: FIG. 1A) 80% PS, 1:72 MSP1D1 to lipids ratio; FIG. 1B) 80% PS, 1:47 MSP1D1 to lipids ratio; FIG. 1C) 50% PS, 1:47 MSP1D1 to lipids ratio, fraction I; FIG. 1D) 50% PS, 1:47 MSP1D1 to lipids ratio, fraction II; FIG. 1E) 20% PS, 1:47 MSP1D1 to lipids ratio. The graphs show the size distribution of the ND with different lipid composition, as characterized by direct measurements from the EM images of ˜200 ND particles diameter. The ND particles with different diameter are separated in four groups and color-coded as follows: white <10 nm diameter, grey between 10.0 and 14.0 nm diameter, dark grey between 14 and 18 nm and black >18 nm. The average diameter and standard deviation are shown for each ND population. Representative class averages (2D class averages of ND particles with similar diameter, as classified by similarity in shape and density/mass distribution by the reference free 2D classification algorithms implemented in EMAN2 (34)) are shown under the graphs.

FIGS. 2A to 2D are graphs that show size exclusion chromatography (SEC) elution profiles and dynamic light scattering (DLS) measurements of the ND at different PS compositions and MPS1D1 to lipids ratio. On the left are the elution profiles of the ND, as obtained from the FPLC. The scale at the bottom of the elution profiles show the diameters (d) of the particles in nm, as calibrated from the elution times of standard proteins with known Stokes radii: thyroglobulin, 8.5 nm; ferritin, 6.1 nm; bovine liver catalase, 5.2 nm; bovine serum albumin, 3.55 nm. On the right are shown the DLS particles distribution from the pooled FPLC fractions. The mean hydrodynamic radius (r) of the ND particles is shown in nm and their polydispersity, as a standard deviation (SD). The mass distribution of each ND population is shown as a percentage of the total mass of particles (y-axis, %). FIG. 2A: phosphatidylserine (PS) 80%, 1:72 MSP1D1 to lipids ratio; FIG. 2B: PS 80%, 1:47 MSP1D1 to lipids ratio; FIG. 2C: PS 50%, 1:47 MSP1D1 to lipids ratio. Two separate populations of ND were pooled into fraction I and fraction II and the DLS measurements were carried out separately for fraction I and fraction II. FIG. 2D: PS 20%, 1:47 MSP1D1 to lipids ratio.

FIG. 3 is a graph that shows data digitized and the area measure with the UCSF Chimera software suite for image visualization and analysis. No Treatment, Nanodisks-(ND), pFVIII, pFVIII-ND. 1=30 seconds, 40=20 minutes.

FIG. 4A is a graph that shows the averaged results shown on in FIG. 3 with the STDEV plotted to one side.

FIG. 4B is a graph that shows the average total volume per condition (over 20 minutes) as shown on FIG. 4A. No treatment, -ND, -pFVIII, -pFVIII-ND. 1=30 seconds, 40=20 minutes.

FIG. 5 is graph that shows the results of an anti-FVIII ELISA titer of rpFVIII (circles) and rpFVIIIND (squares). FVIII deficient mice were injected every 7 days with 2 μg/mouse recombinant porcine FVIII alone or porcine FVIII-ND complex. Each mouse received 4 doses, and plasma was obtained one week after the final dose. Serial dilutions (1:2) of plasma were added to ELISA plates coated with porcine or human FVIII, and the highest plasma dilution that produced a reading of ≧0.2 OD over background at 450 nm were reported as endpoint titers.

FIG. 6 shows a far UV-CD spectra and secondary structure deconvolution of MSP1D1 alone and when assembled in ND at different lipid composition.

FIGS. 7A and 7B are EM micrographs of negatively stained Y branched and side-attached nanodisk (ND) stacks adsorbed on amorphous carbon assembled at MSP1E3D1 to lipids ratio of 1:150 after 60 minutes incubation with 10 mM CaCl₂. The inset shows double and triple ND stacks formed bellow 5 mM CaCl₂. Scale bar is 10 nm. The protein-lipid densities are in white. FIG. 7B. Cryo-EM micrographs of the ND stacks in amorphous ice (vitreous water) after 60 minutes incubation with 10 mM CaCl₂. The protein-lipid densities are in black.

FIG. 8. Electron micrographs of negatively stained ND and FVIII-ND complexe on amorphous carbon. The insets show magnified views of the square areas. Scale bar is 10 nm. Representative 2D class averages from selected ND and FVIII-ND particles boxed at 180×180 pixels at 2.9 Å/pix and masked with a radial mask.

FIGS. 9A and 9B show the activity of FVIII in solution and bound to nanotubes (LNT) and ND containing 80% PS and scaffolding protein (MSP1D1) to lipid of 1:47 as measured by the activated partial thromboplastin time (aPTT) assay against FVIII deficient human plasma. FIG. 9A. Standard curve for Factor Assay Control plasma (FACT) containing 1 U/ml human FVIII. FIG. 9B. Activities of human and porcine FVIII in solution and when bound to LNT and ND. The LNT and ND were mixed in an excess of 10 times to the FVIII to secure that all FVIII molecules are in a membrane-bound state.

FIGS. 10A and 10B are Cryo-EM micrographs (1024×1024 pixels @ 1.4 Å/pix) of porcine FVIII-LNT (FIG. 10A) and FVIIIa-LNT (FIG. 10B) helically organized on LNT. The insets show the Fourier transforms. FIG. 10C shows a SDS-PAGE of porcine FVIII and pFVIIIa, showing the trimeric nature of the FVIIIa molecule. The molecular weight of the standard proteins is in kDa.

FIG. 11 shows the size distribution of PS-GC-ND assembled from PS:GC=4:1 ratio and two MSP: MSP1D1 and MSP1E3D1 at different MSP to lipid ratio. Left. EM micrographs of negatively stained ND adsorbed on amorphous carbon. Scale bar 50 nm. Right. Size distribution graphs as measured from the NS-EM micrographs.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Factor VIII is a multidomain plasma glycoprotein, which when activated serves as a cofactor to serine protease FIXa within the membrane-bound FVIIIa-FIXa complex. Human FVIII (hFVIII) polypeptide chain consists of 2332 amino acids organized in six domains: A1-A2-B-A3-C1-C2. After expression and purification FVIII forms a mixture of heterodimers of a heavy chain (HC) of the A1-A2 domains with parts of the B domain and a constant length light chain (LC) of the A1-C1-C2 domains. The LC and HC are non-covalently linked via divalent Ca²⁺ cation(s). FVIII is activated by thrombin resulting in proteolytic cleavage at Arg372 that separates the A2 and A1 domains. Activated FVIII (FVIIIa) is a heterotrimer composed of non-covalently linked A1, A2 domains and the LC. The activity of FVIIIa in vitro is quickly abolished by spontaneous dissociation of the A2 domain (˜6 minutes), which is only weakly attached to the rest of the molecule (Kd˜500 μM). Recombinant porcine FVIII-BDD (pFVIII) has 84% sequence identity to hFVIII and is expressed at much higher yield (10 to 14 fold). The active form—pFVIIIa, is significantly more stable in vitro especially in its membrane-bound form and assembles equally with human FIXa on PS rich membranes. As taught herein, the well-characterized FVIII-BDD forms can be used to show how differences in sequence can modulate the macromolecular interactions governing the membrane-bound assembly of these proteins and their complexes with FIXa.

The macromolecular organization of the FVIII domains in its membrane-bound form and their synchronization are the driving force behind FVIII activity and the assembly of the FVIIIa-FIXa complex on the activated platelet surface. The presence of multiple membrane-binding sites at the FVIII-LC interface is due to the flexibility of the molecule and specifically the short link between the C1 and C2 domains lacking a stable hydrophobic macromolecular interface. While a 4 Å resolution structure of FVIII in solution has been resolved by X-ray crystallography, a different membrane-bound domain organization has been resolved. The structural basis of FVIII activation and the FVIIIa-FIXa complex assembly strongly depend on the domain organization and are not completely understood to date. Understanding the macromolecular forces driving the FVIII activation and the FVIIIa-FIXa complex assembly on a PS-rich membrane surface is fundamental for the successful design of membrane-mimetics modulating blood hemostasis at the FVIII level.

Nanodiscs (ND) are lipid bilayer membrane patches held by amphiphilic scaffolding proteins (MPS) of ˜10 nm in diameter. Nanodiscs have been developed as lipid nanoplatforms for structural and functional studies of membrane and membrane associated proteins. Their size and monodispersity have rendered them unique for electron microscopy (EM) and single particle analysis studies of proteins and complexes either spanning or associated to the ND membrane. Binding of blood coagulation factors and complexes, such as the Factor VIII (FVIII) and the Factor VIIIa-Factor IXa (intrinsic tenase) complex to the negatively charged activated platelet membrane is required for normal hemostasis. ND were specifically designed to bind FVIII at close to physiological conditions. The binding of FVIII to the negatively charged ND rich in phosphatidylserine (PS) was followed by electron microscopy at three different PS compositions and two different membrane scaffolding protein (MSP1D1) to lipid ratios. These results show that the ND with highest PS content (80%) and lowest MSP1D1 to lipid weight ratio (1:47) are the most suitable for structure determination of the membrane bound FVIII by single particle EM. These results also show that FVIII 3D reconstruction as bound to PS containing ND is suitable for the optimized ND for structural studies by EM. Further assembly of the activated FVIII form (FVIIIa) and the whole FVIIIa-FIXa complex on ND, followed by EM and single particle reconstruction can help to identify the protein-protein and protein-membrane interfaces critical for the intrinsic tenase complex assembly and function.

Lipid Nanotubes (LNT) were developed for helical organization of membrane-associated proteins allowing near atomic structure determination by Cryo-EM. The size and monodispersity of ND make them more desirable for structural studies of macromolecular complexes than LNT due to challenges in achieving sufficient helical organization for high-resolution Cryo-EM. LNT have the advantage to be morphologically similar to the filopodia formed upon activation of platelets—the biological PS rich membrane surface for the assembly of FVIII and its complexes. PS-rich ND have been shown to be suitable for the assembly of the serine protease Factor VIIa (FVIIa) in the absence and presence of its co-factor, Tissue Factor (TF). The structure and biochemistry however of the co-factors: TF and FVIIa is radically different, which is reflected in the rate of proteolytical activation by the respective proteases: slow for FVIIa-TF and fast for FVIIIa-FIXa membrane-bound complex. Therefore, optimizing ND and LNT for the assembly and Cryo-EM structural studies of the membrane-bound co-factor proteins—FVIIIa and FVa and their complexes with the respective serine proteases—FXa and FXa the FVIIIa-FIXa on membrane nano-platforms mimicking the activated platelet surface is novel.

Reagents. HEPES sodium salt, Na cholate and MSP1D1 scaffolding protein were obtained from Sigma (Sigma-Aldrich, St. Louis, Mo., USA). Phospholipids-phosphatidylserine—PS (1,2-dioleoyl-sn-glycero-3-phospho-L-serine, sodium salt) and galactosylceramide—GC (D-galactosyl-β-1,1′ N-nervonoyl-D-erythro-sphingosine, d18:1/24:1) were obtained from Avanti Polar Lipids, Inc (Alabaster, Ala., USA). All buffer solutions—HBS (NaCl 150 mM, HEPES 20 mM, pH 7.4) and HBS-Ca (NaCl 150 mM, HEPES 20 mM, CaCl2 5 mM, pH 7.4) were filtered through 0.22 μm MILLEX® GP filter (Millipore, Carrigtwohill, Co. Cork, Ireland).

Nanodiscs Preparation. Nanodiscs (ND) were prepared following the procedure described in (31) with some modifications. Briefly, PS and GC were dissolved in chloroform and mixed in desired weight ratio. The chloroform was evaporated under Argon and the lipids reconstituted in HBS with 26 mM of Na cholate, warmed up to 70° C. and sonicated for 15 minutes (min). The MSP1D1 was reconstituted in HBS with 15 mM Na cholate. The MSP1D1 and lipids were mixed in the desired molar ratios with a final lipid and Na cholate concentration of 6 mM and 20 mM, respectively, and incubated for 1 hour (h) at 37° C. The Na cholate was removed by adding 1 g of Bio-Beads SM-2 per 1 ml of mix and incubation for 4 hours at room temperature. The Bio-Beads were removed by centrifugation and the ND suspension was further purified by fast protein liquid chromatography (FPLC) through a Superdex 200 HR 10/30 column equilibrated in HBS solution. The fractions corresponding to the ND peak were pooled out and concentrated in Vivaspin with 10k cut-off membrane.

Porcine Factor VIII Purification. B-domain deleted porcine FVIII was expressed in BHK cells and purified as previously described (19, 28). The FVIII activity was estimated by the one-stage—activated partial thromboplastin time (aPTT) clotting assay and stored in HBS-Ca buffer at −80° C. For the FVIII-ND experiments the FVIII was concentrated through 0.22 μm MILLEX® GP filter (Millipore, Carrigtwohill, Co. Cork, Ireland). The protein concentration was estimated with a Nanodrop Spectrophotometer ND-1000 (Thermo Fisher Sci Inc, Waltham, Mass., USA) and calculated using the molar absorption coefficient at 280, ε (280)=258965 M−1cm−1, calculated from the known tyrosine, tryptophan and cysteine content of FVIII (32).

aPTT Activity Test. To measure the activity of FVIII the samples were diluted in EMS buffer to a concentration of FVIII within the range of standard curve and incubated with FVIII deficient plasma (<1% activity, George King Bio-Medical, Inc.) and aPTT reagent (TriniCLOT Automated aPTT, Tcoag Ireland Limited, Ireland) for 4 minutes at 37° C. before adding the CaCl₂ and measuring the clotting time. For the FVIII-ND activity tests, the ND were mixed in excess of 10 times to the FVIII and incubated 10 minutes at room temperature, and a control ND sample was also prepared at the same ND concentration as the FVIII-ND sample. All measurements were repeated three times and the averaged time was used for the evaluation of FVIII activity against the Factor Assay Control Plasma (FACT, 1 U/ml of FVIII activity) (George King Bio-Medical, Inc.) as previously described (28).

Sodium dodecyl sulfate polyacrylamide gel (SDS-PAGE) electrophoresis. READY GEL® Tris-HCl Gels (4-15%) (Bio-Rad, Hercules, Calif., USA) were run according to standard protocol (33). Protein sample at concentration 2 μg/well were reduced with 5% β-mercaptoethanol in Bio-Rad Laemmli Sample buffer at 94° C. for 5 minutes, loaded onto gels with Bio-Rad Prestained Standards Broad Range and run at 115 V for 2.5 hours. The gels were stained with GELCODE® Blue Stain Reagent (Thermo Scientific, Rockford, Ill., USA) and de-stained with water.

Dynamic Light Scattering (DLS). All DLS measurements were carried out with a Zetasizer μV particle analyzer (Malvern Instruments Ltd, Malvern, UK). The measurements were carried out in a 2 μl volume quartz cuvette at 21° C. at a light-scattering detection angle of 90°. Diameter of particles were calculated from the intensity autocorrelation curve with the assumption that the particles are spherical; with regard to real particles that often are non-spherical, dynamic and solvated in solution, the calculated diameter represents the apparent size of the dynamic hydrated/solvated particle—hydrodynamic diameter. Each measurement represented 10 consecutive single measurements each of averaged 13 runs. The final particle size and mass distribution were compiled by averaging the data from 10 independent measurements. All calculations were done by Zetasizer Software.

Electron Microscopy (EM). Sample preparation. The NDs were diluted down to 0.005 mg/ml MSP1D1 protein concentration in HBS buffer. The FVIII-ND samples were prepared by mixing equal volumes of FVIII and ND solutions to obtain 1:1 molecular FVIII to ND ratio incubated 5 minutes at room temperature (21° C.) and diluted as for the ND in HBS-Ca buffer. Five μl of the sample were deposited onto freshly carbon-coated and plasma treated hexagonal grid (300 mesh, Ted Pella, Redding, Calif., USA) and stained by 1% Uranyl acetate (UA).

Data Collection. Digital electron micrographs were recorded with a 4096×4096 pixel Ultrascan CCD camera (Gatan, Inc., Pleasanton, Calif., USA) at low electron dose conditions (<20 electrons/Å 2.s) and a final magnification of 56,400×, attached JEM2100-LaB6 transmission electron microscope (JEOL, Ltd) operated at 200 kV.

Single-Particle Analysis. The size of the ND was evaluated with the Digital Micrograph software (Gatan Inc). 2D analysis and single particle reconstruction of the ND and FVIII-ND particles was carried out with the EMAN2 scientific image-processing suite (34) with the e2projectmanager.py graphic user interface (GUI) calling the individual EMAN2 programs required for the single particle analysis. The ND and FVIII-ND particles were boxed at 100×100 and 180×180 pixels (2.9 Å/pix), respectively, corrected for the contrasts transfer function (CTF), combined and classified by similarity in size (ND diameter) and organization of the FVIII molecules bound to the ND surface with the iterative reference free alignment algorithm: e2refin2d.py implemented in EMAN2 (34). Particles from the same classes were combined in a new datasets for further 3D reconstruction of the ND and the FVIII-ND complexes.

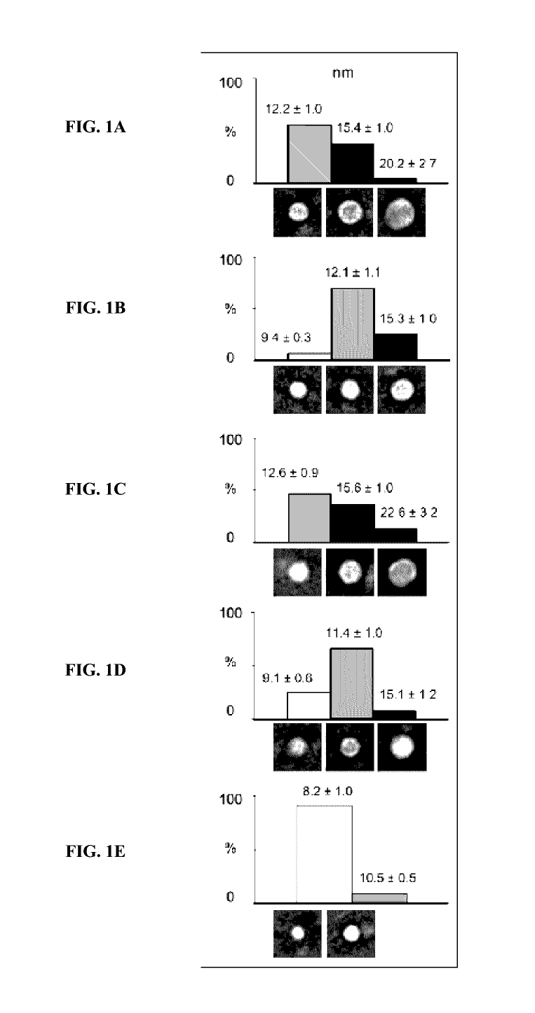

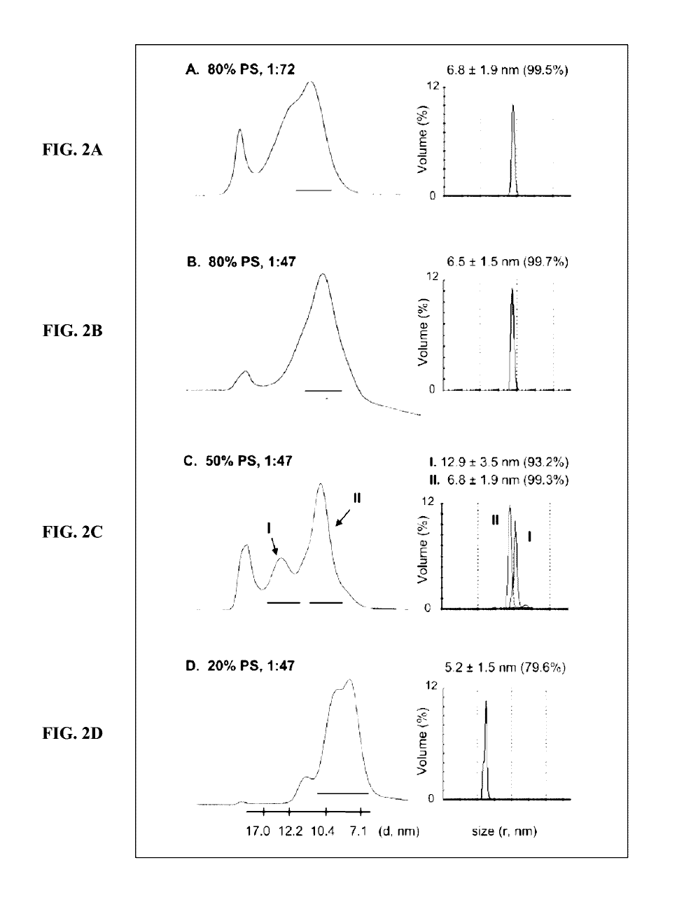

The lipid composition of the ND was designed based on the inventor's previous work with lipid nanotubes (LNT) and how their lipid composition, different GC and PS content affects the FVIII membrane bound organization. A theoretical MSP1D1 protein to lipids ratio was calculated, which was equal to 1:72 and assembled the GC based ND with 80% PS content, as this concentration was proven to be the most favorable for the organization of membrane-bound FVIII on LNT. As the ND at MSP1D1 to lipid ratio of 1:72 proved to be quite heterogeneous (FIG. 1A, FIG. 2A) this ratio was lowered to determine empirically, which MSP1D1 to lipid ratio is most suitable for the ND system taught herein. The most homogenous ND population was obtained at MSP1D1 to lipid ratio of 1 to 47 (FIG. 1B, FIG. 2B) and this ratio was used to further determine the effect of the PS concentration on the ND assembly and shape. The same range of PS concentrations was selected based on the inventor's previous GC based LNT studies: 20%, 50% and 80% PS (FIGS. 1A to 1E, FIGS. 2A to 2D) (35). For the purpose of structure determination by EM it was important to evaluate the shape of the ND particles and size distribution after the assembly process. The size exclusion chromatography (SEC) elution profile cannot provide an exact value for the particles' size distribution due to the averaging effect of this technique (36). Next, the ND size and shape was evaluated by negatively stained electron microscopy (NS-EM) after the FPLC filtration (FIGS. 1A to 1E, FIGS. 2A to 2D). Up to 100 micrographs were collected from the ND at different lipid composition. Twenty micrographs of the best negatively stained regions (homogenously stained and distributed ND suitable for boxing by EMAN2) were further selected for single particle analysis (SPA). The negatively stained ND showed preferable top-view orientation when adsorbed to the amorphous carbon. Less than 10% of the ND particles had side view orientation (Table 1).

TABLE 1
ND particles selection from the NS-EM images. The percentage (%) of
side-views (third column) was estimated by assessing the amount of
particles classified in the class averages showing pronounced side-views
as portion of the total particle population (middle column). *—due to the
small size (~8 nm) of the 20% PS ND, the class averages of
side-views were difficult to distinguish from top-views.

| | Total amount of boxed | |
|---|---|---|
| Sample | particles | % of side-views |
| PS 80%, MSP:lipid 1:72 | 8485 | 8% |
| PS 80%, MPS:lipid 1:47 | 6724 | 5% |
| PS 50%, MPS:lipid 1:47, I | 2266 | non-significant |
| fraction | | amount |
| PS 50%, MPS:lipid 1:47, II | 6059 | 9% |
| fraction | | |
| PS 20%, MPS:lipid 1:47 | 7073 | —* |

The size of each particle was determined by measuring directly the diameter of the ND from the acquired digital EM micrographs (summarized in FIGS. 1A to 1E). The size evaluation of the ND particles by EM showed that the 80% PS lipid composition and MSP to lipid ratio of 1:47 gave a highest population of homogenous ND with a diameter of ˜12 nm. The discs assembled at 1:72 MSP1D1 to lipid ratio and same lipid composition (PS=80%) were more heterogeneous, showing a predominant population with a larger diameter of ˜15 nm (FIG. 1A, 1B). This higher heterogeneity at 1:72 MSP1D1 to lipid ratio was also confirmed by the SEC where the elution profile of discs at 1:72 ratio, as opposed to 1:47 ratio, had a shape characteristic for not optimal MSP1D1 to lipid ratio (FIG. 2A, 2B) (37). For this reason, all further ND assemblies with different PS content were carried out at 1:47 MSP1D1 to lipid ratio. The inventors followed the same NS-EM and SEC evaluation procedure for the ND assembled at 50% and 20% PS concentration (FIG. 1C-E, FIG. 2C, 2D). The SEC elution profiles for all ND assembled at different PS composition and MPS1D1 to lipid ratio were distinct from each other and corresponded to the direct measurements from the EM micrographs, and the calculated 2D class averages (FIGS. 1A to 1E). The SEC elution profiles showed one broad peak for the ND containing 20% PS centered at ˜8 nm diameter and two distinct peaks for the ND containing 50% PS centered at ˜10 nm and ˜12 nm, respectively (FIGS. 2A to 2D). The size and distribution of the ND fractions from the SEC eluted peaks were also evaluated by DLS (FIGS. 2A to 2D).

From the NS-EM measurements, the ND were separated into size groups with bandwidth 4 nm: 8±2 nm, 12±2 nm, 16±2 nm, and >18 nm. The average size of each ND group was calculated from 200 particles, selected from several micrographs. The number of particles in each group was plotted as percentage from the total amount of measured particles (FIGS. 1A to 1E). To evaluate further the ND size distribution at different lipid compositions and MSP1D1 to lipid ratio, individual ND particles were selected from several EM micrographs from each condition and classified the particles in separate classes according to similarities in shape and dimensions employing the reference free 2D classification algorithms implemented in EMAN2 (34). The calculated 2D class averages confirmed the size obtained by direct measurement of the particles on the EM digital micrographs (FIGS. 1A to 1E). The values for the ND diameter obtained by all three methods: SEC, DLS and NS-EM were reasonably consistent; however, there was a difference in the size (average diameter) estimation for each ND population when measured with a different method (FIGS. 1A to 1E, FIGS. 2A to 2D). This can be explained with the fact that SEC and DLS techniques are not very accurate in distinguishing ND particle populations in the ˜14-20 nm diameter range. Therefore, NS-EM was considered to be the most accurate way to measure the ND size and the values obtained by this technique were the one employed for further structure determination (data not shown).

The effect of different size and lipid compositions on the FVIII membrane-bound organization was followed by adding recombinant porcine FVIII in HBS-Ca buffer to the ND with different PS content at 1:1 molecular ratio. The effect of the PS concentrations on the FVIII membrane-bound organization was followed by NSEM of the negatively stained FVIII-ND complexes adsorbed on amorphous carbon film. The EM micrographs of the FVIII-ND assemblies showed that the FVIII molecules attach to the ND surface in multiple configurations (data not shown). The number of FVIII molecules attached to one ND, as well as the distribution of ND with FVIII molecules in similar organizations was unambiguously influenced by the lipid composition and specifically the PS content of the ND. To confirm this observation FVIII-ND particles were boxed from the NS-EM micrographs and calculated 2D averages by classifying the particles in separate classes with the reference free alignment algorithms implemented in the EMAN2 software (34, 38). The ND with 80% PS lipid composition and MPS1D1 to lipid ratio of 1:47 showed the most homogenous population of FVIIIND complexes with one FVIII molecule bound to one or both ND sides. This population of FVIII-ND complexes also showed a better proportion between the ND and bound FVIII volumes, compared to the ND with 20% PS lipid composition. The 20% PSND are not large enough to be unambiguously differentiated from the membrane-bound protein. Additionally, due to the less accessible lipid surface of the 20% PS ND, the optimal organization of the FVIII molecules were disturbed and less homogenous when bound to the ND surface, thus making them difficult for further structural analysis (data not shown).

To further test the efficacy of the 3D reconstruction algorithms on the best ND and FVIII-ND data sets more NS-EM micrographs were collected with ND at 80% PS concentration and MPS to lipid ratio of 1:47. To calculate the ND 3D structure, 6,724 ND particles were boxed at 100×100 pixels (2.9 Å/pixel). After several cycles of reference free alignment 2D classification carried out within EMAN2 (34), a final particle set of 1,309 ND was selected consisting of ND with a diameter of 13±1 nm. The ND 3D structure was further calculated with the single particle analysis (SPA) 3D reconstruction algorithms implemented in EMAN2 (34) and filtered at 25 Å resolution. The ND with 80% PS and MSP1D1 to lipid ratio of 1:47 showed a donut shape corresponding to the lipid bilayer nanodiscs (data not shown). Representative 2D class averages from the 1309 ND particles boxed at 100×100 pixels (2.9 Å/pix). The number of particles in each class is indicated. Corresponding 2D projections from the 3D volume of the GC-ND at 80% PS and MSP1D1 to lipid ratio of 1:47 (data not shown) obtained from the same data set with the e2refine.py algorithms implemented in EMAN2.

As the most homogenous FVIII membrane-bound organization was observed with the ND at 80% PS and MSP1D1 to lipid of 1:47 more EM micrographs were collected at these conditions and boxed 13,022 FVIII-ND particles at 180×180 pixels (2.9 Å/pixel) for further image processing. Further selection of FVIII-ND particles with FVIII bound only to one side yield a data set of 4,757 particles, which was further narrowed down to 2,791 particles by selecting FVIII-ND particles with ND diameter of 14±1 nm. For the 3D reconstruction a final data set of 1,162 FVIII-ND particles was obtained by manual selection of the best and evenly stained complexes, which gave a reconstruction similar to the one previously calculated in (30), filtered to 25 Å resolution (data not shown). In this new FVIII-ND 3D reconstruction, the two membrane-bound FVIII molecules are better differentiated and the membrane-bound interface is more clearly defined. The correctness of the 3D reconstructions was further confirmed by the very good correlation between the 2D class averages from the final data sets (ND and FVIII-ND particles) obtained from the selected EM-2D images by the 2D classification algorithms implemented in EMAN2 (data not shown), and the corresponding 2D projection from the ND and FVIII-ND 3D volumes calculated by projecting the ND and FVIII-ND 3D volumes at equidistant Euler angles (orientations) on a 2D plane (data not shown). Representative 2D class averages from the 1162 FVIII-ND particles boxed at 180×180 pixels (2.9 Å/pix). The number of particles in each class is indicated. Corresponding 2D projections from the FVIII-ND 3D volume (data not shown) obtained from the same data set with the e2refine.py algorithms implemented in EMAN2.

The activity of FVIII bound to ND with 80% PS concentration and MSP1D1 to lipid of 1:47 was evaluated with the aPTT test. The FVIII+ND activity was compared with the FVIII activity in solution at equal amount of FVIII to confirm the functionality of the resolved FVIII membrane-bound structure (Table 2).

TABLE 2
Activity of porcine FVIII and FVIII-ND as measured with the aPTT test.
The porcine FVIII-ND sample was prepared with ND at 80% PS
concentration and 1:47 MSP1D1:to lipid ratio.

| | | U/ml | |
|---|---|---|---|
| Samples | Clotting time (s) | of diluted sample | Total activity, U/ml |
| FVIII | 58.1 | 0.19 | 1530 |
| FVIII + ND | 57.7 | 0.20 | 1777 |

Lipid ND have been proven to be suitable for the assembly and functional studies of blood clotting factors and complexes (17, 39). Nanodiscs at 100% PS (POPS) concentration have been previously utilized for the assembly and study of the Factor VIIa (FVIIa)-Tissue Factor (TF) complex (17). It has been also reported that larger ND might be required for the assembly of the Prothrombinase complex: between the Factor Va and Factor Xa, which is highly homologous in mass and possibly membrane-bound organisation to the intrinsic tenase complex assembled from the FVIIIa and FIXa on the same activated platelet membranes (39).

In this study, galactoceramides (GC) were introduced as the base lipid for the ND assembly to monitor the effect of different PS concentration on the membrane-bound organization and assembly of the FVIII. Due to the high transition temperature (Tₘ) of GC (Tₘ GC 24:1 utilized in this study is ˜67° C.), they have a complex miscibility with phospholipids in bilayers, as they present in a gel phase at physiological conditions. ND assembly is optimal above the Tₘ of the lipid mixture, which in the case of the GC is too high and might denature the scaffolding protein, a critical temperature for Nanodiscs is 55° C. (40). For this reason the inventors did not attempt to assemble pure GC ND and followed ND assembly at various PS concentrations (DOPS Tₘ ˜11° C.), which allowed proper assembly of the ND at room temperature. GC also contributes to the ND rigidity, making them more amenable for structural studies by EM. Sphingolipids such as GC are natural lipids, which account for ˜30% of the activated platelet membranes lipids. The aPTT clotting tests showed that the activity of FVIII when bound to the ND is comparable to the activity of FVIII without ND, which confirms the physiological compatibility of the GC based ND taught herein.

Finally, GC is also the essential lipid for the lipid nanotubes (LNT) assembly used for the Cryo-EM studies of membrane-bound FVIII helically organized on GC based LNT containing 80% PS. Thus, utilizing different nanotechnologies with the same lipid composition for structural studies of FVIII allows definition of the functional membrane-bound organization of FVIII and its significance for the intrinsic tenase complex assembly in the propagation phase of coagulation.

The NS-EM approach employed for the evaluation of ND at different lipid compositions and MSP1D1 to lipid ratios is the most direct visualization method to estimate the size of lipid nanostructures adsorbed on amorphous carbon film at near to physiological conditions. This approach was applied to select the best ND lipid composition and FVIII-ND assembly conditions, which can be further used for high-resolution structure analysis by Cryo-EM. As NS-EM has significantly higher contrast than Cryo-EM this approach is more efficient for characterizing the effect of multiple conditions, such as lipid composition that can yield highly uniform by size and shape ND, required for structure analysis. The most homogenous populations of ND have been selected by this method to further characterize the FVIII membrane-bound organization. This population consisted of FVIII-ND assembly showing ND with the same shape and diameters, and FVIII bound to only one side. Further single particle analysis (SPA) of this most homogenous FVIII-ND population was used to generate an initial membrane-bound FVIII-ND model (˜25 Å resolution). This FVIII-ND 3D reconstruction will be further used as a template to refine higher resolutions structures (<10 Å resolution) from the Cryo-EM data (data not shown).

Calculating intermediate resolution structures of ND at different lipid composition and of FVIII-ND at different FVIII configurations is also useful to evaluate the overall geometrical properties of the ND and membrane-bound FVIII molecules. The values of the molecular area of the PS and GC lipids are estimated to be ˜65 Å2 and ˜40 Å2, respectively, at physiologically relevant pressure of 30 mN/m (42, 43). The MSP1D1 is a deletion (L′1-54) mutant of human Apolipoprotein A-I with attached 7-His-tag and TEV protease site of 189 amino acids (aa) and molecular weight ˜25 kDa (31). Since each turn of the α-helix comprises 3.6 amino acids (aa) and one aa corresponds to 1.5 Å increment along the helical axis, one MSP1D1 molecule can form a circle of 90.3 ∈ of diameter. Taking into account that the diameter of an α-helix with the side chains is ˜10 Å, the MSP1D1 can form a donut-like structure with inner diameter of 80 Å and outer of 100 Å, which will contain ˜100 lipid molecules, assuming ˜50 Å2/molecule area (37). In this case, the ND from the FVIII-ND complexes have a larger diameter of ˜130 Å, which can accommodate up to 160 lipid molecules on each side of the ND bilayer. From previous studies of FVIII organized in 2D crystals on PS containing monolayers (44, 45) the inventors recognized that one membrane-bound FVIII molecule covers ˜60 lipids molecule. Thus, combining the information from the FVIII-ND 3D reconstructions with the inventor's previous FVIII-LNT data (above) it is now possible to prove that the FVIII membrane-bound molecules can organize as dimers not only on the LNT, but also on the ND surface. Such dimeric organization has been previously observed with membrane bound Factor Va and Factor IXa organized in membrane-bound 2D crystals (46, 47). The presented ND and FVIII-ND reconstructions in this work are a first direct visualization of the FVIII membrane-bound structure at 25 Å resolution without imposed order.

Organizing membrane-associated proteins in 2D and helical crystals can be a time consuming and challenging task, both experimentally and from a structural point of view, as 2D crystals require titling in the microscope for 3D reconstruction and helical analysis requires perfect helices for proper indexation. The presented in this work approach aims to select homogenous FVIII-ND particle sets suitable for high-resolution single particle analysis where neither tilting nor order is required. This methodology will be also ideal for the study of the membrane-bound organization of whole lipid and solution conditions.

Thus, the present invention includes an optimized ND-based membrane platform for structural studies of membrane-bound coagulation proteins and their complexes. First, FVIII-ND complexes were characterized, yielding reliable 3D reconstructions at 25 Å. The ND taught herein can also be to study the assembly of the whole FVIIIa-FIXa complex, by varying the type of MSP proteins and the MSP to lipids composition. These results with porcine FVIII and MSP1D1 show that this method can lead to high-resolution structure determination by combining Cryo-EM, SPA and ND technologies.

### EXAMPLE 2

Hemophilia A is a congenital bleeding disorder resulting from defective or deficient factor VIII. The active form of Factor VIII is the cofactor to the serine protease Factor IXa (FIXa) in membrane-bound intrinsic tenase (FVIIIa-FIXa) complex. The assembly of the FVIIIa-FIXa complex on the activated platelet surface in the propagation phase of coagulation amplifies FXa and thrombin generation more than five orders of magnitude required for successful blood clotting.

The inventors sought to optimize and characterize the role of lipid nanodiscs for the Factor VIII function in vivo and to test the implication of FVIII(a) and Factor IX(a) binding to nanodiscs for successful application to treat Hemophilia.

Methods: Purification and expression of Factor VIII (FVIII). SDS gels and aPTT test. Nanodiscs (ND) and FVIII-ND assembly and characterization by EM and aPTT test. In vivo test of the FVIII-ND complexes in hemophiliac mice and time resolved snip-tail tests. Antibody tests of the FVIII-ND complexes in hemophiliac mice and ELISA.

Results: Fully active Factor VIII was assembled on lipid nanodiscs. The FVIII-ND complexes showed a pronounced procoagulant effect, which was stronger than FVIII alone, when injected in hemophilic mice. The ND also showed a procoagulant effect, however the FVIII-ND effect was not additive, suggesting a synergistic effect of the FVIII-ND complexes on the clotting process in hemophilic mice.

Overall binding the FVIII to ND prior injection in hemophilic mice, improves significantly the therapeutic function of the protein. This can be a significant step towards a new approach to modulate coagulation at the level of the membrane-bound Factor VIII and the whole intrinsic tenase (FVIIIA-FIXa) complex.

A time course of clotting from tail-snip tests of hemophiliac mice after administrating of: lipid nanodiscs (ND), porcine FVIII (pFVIII) and pFVIII bound to ND (pFVIII-ND) was conducted, with No tx is no treatment. Each drop correspond to the amount of blood blotted to a hardened filter paper for 10 second each 20 seconds after the tail snip (data not shown).

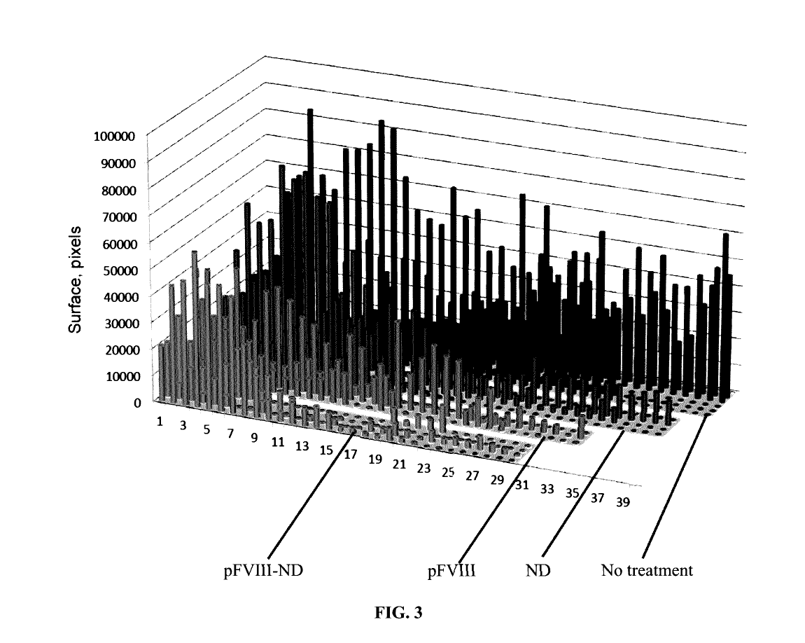

FIG. 3 is a graph that shows the tail-snip tests of hemophiliac mice after administrating of: lipid nanodiscs (ND), porcine FVIII (pFVIII) and pFVIII bound to ND (pFVIII-ND) was conducted, with No tx is no treatment data after being digitized and the area measured with the UCSF Chimera software suite for image visualization and analysis. No treatment, -Nanodisks (ND), -pFVIII, -pFVIII-ND. 1=30 seconds, 40=20 minutes.

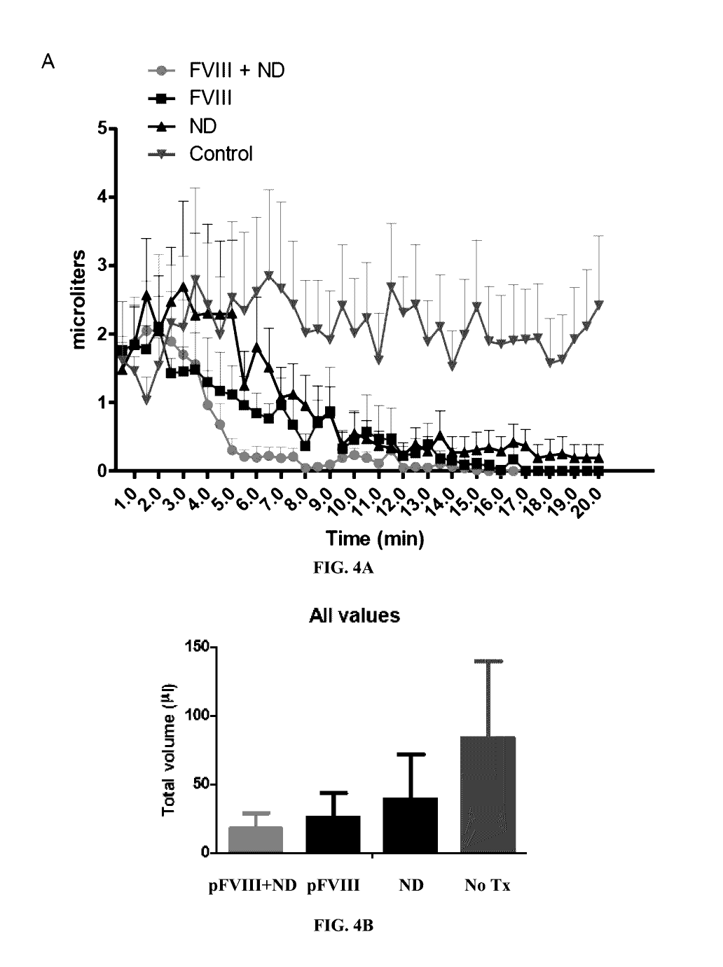

FIG. 4A is a graph that shows the averaged results shown on slide #3 with the STDEV plotted to one side. FIG. 4B is a graph that shows the average total volume per condition (over 20 minutes) as shown on FIG. 4A. No treatment, Nanodisks (ND), -pFVIII, pFVIII-ND. 1=30 seconds, 40=20 minutes.

Immunogenicity of rpFVIII and rpFVIII-ND in FVIII-KO mice. To test if assembly of rpFVIII with ND affected its immunogenicity, the inventors injected FVIII-KO mice 433 with 2 μg/mouse of rpFVIII alone or rpFVIII ND. As shown in FIG. 6, the injection of combined rpFVIII-ND resulted in slightly, but not significantly (p=0.090) increased plasma levels of rpFVIII specific IgG antibody (average titer of 28 v/s 210). The anti-FVIII titers produced in FVIII-KO mice in response to injection of rpFVIII-ND complexes were comparable to those reported in other studies following repeated doses of recombinant FVIII [46,47].

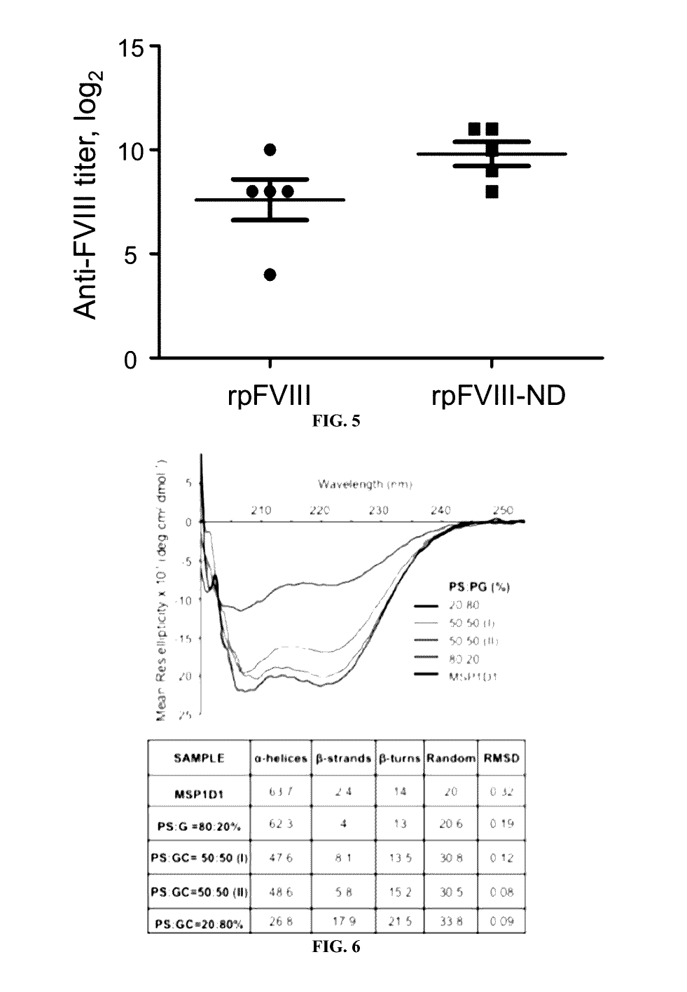

FIG. 5 is graph that shows the results of an anti-FVIII ELISA titer of rpFVIII (circles) and rpFVIIIND (squares). FVIII deficient mice were injected every 7 days with 2 μg/mouse recombinant porcine FVIII alone or porcine FVIII-ND complex. Each mouse received 4 doses, and plasma was obtained one week after the final dose. Serial dilutions (1:2) of plasma were added to ELISA plates coated with porcine or human FVIII, and the highest plasma dilution that produced a reading of ≧0.2 OD over background at 450 nm were reported as endpoint titers.

As shown herein, by altering the lipid composition and geometry of ND the present invention can be used as part of an effective drug delivery systems for clotting factors and complexes that can modulate blood coagulation and can be personalized to a patient's needs

Lipid ND as drug delivery systems for membrane-bound clotting factors. The lipid composition and geometry of the PS-rich ND were developed to bind clotting factors and complexes at close to physiological conditions. The assembled FVIII-ND complexes are suitable for biophysical, structural and functional studies. Further changes in the lipid composition, type of MSP and the MSP to lipids ratio can be made to optimize for drug delivery and structure determination of membrane-bound clotting proteins by Cryo-EM.

Galactosylceramide (GC) lipid molecules have the ability to self-organize into LNT in aqueous solutions. The assembled PS (DOPS) rich GC-ND nanodisks and nanotubes are suitable for the assembly and structure determination of membrane-associated clotting proteins and specifically FVIII using PS rich GC-LNT complexes. To characterize and optimize the ND best suited for the proposed research the inventors developed a methodology by combining effectively negatively stained (NS)-EM studies with biophysical methods, such as dynamic light scattering (DLS), circular dichroism (CD) and small angle X-ray scattering (SAXS).

Nanodiscs (ND) assembly at different lipid composition, MSP and MSP to lipid ratio. MSP are modified ApoA1 proteins that are almost exclusively helical and amphipathic, forming antiparallel belt-like assemblies stabilizing the edge of the ND lipid bilayer. The ND was first assembled followed the lipid composition of the LNT (PS:GC=80:20%) the inventors optimized for helical organization of membrane-associated FVIII. These nanodiscs were then successfully characterized by size exclusion chromatography (SEC), dynamic light scattering (DLS) and by circular dichroism (CD) the ND assembled with MPS1D1 at three PS concentrations: 20%, 50%, 80% and two different MSP1D1 to lipids ratio 1:47 and 1:72. The type of MSP and the MSP to lipids ratio affects both the homogeneity and size distribution of the ND population. The interplay between the type of MSP and the MSP to lipid ratio are essential for the ND monodispersity and size distribution. The ND with a lipid composition of 80% PS and 20% GC showed to have good biochemical, biophysical and EM results. This ND population has the most homogeneous distribution of ND with diameter of ˜12 nm when the lipids are assembled with MS1D1 at ˜1:47 protein: lipid ratio and is best suited for structural studies of membrane-bound FVIII. The CD spectra for the MSP1D1 in these studies correspond to other known spectra confirming that the ND assembled at lipid composition of PS:GC=80:20% and MSP1D1 to lipids ratio of 1:47 showed no changes in the CD spectra (FIG. 6). FIG. 6 shows a Far UV-CD spectra and secondary structure deconvolution of MSP1D1 alone and when assembled in ND at different lipid composition. The CD data also points to the lipid composition as an important parameter for the ND size distribution and homogeneity at given MSP and MSP to lipids ratio, contrary to the results published for purely phosphatidylcholine (PC) or PC mixtures.

Table 3 is a summary of the biochemical and biophysical characterization of ND
assembled with two different MSP at constant lipid composition of 20% GC and
80% PS at different MSP to lipids (Li) ratios.

| MEASUREMENT METHOD | | | | | |
|---|---|---|---|---|---|
| | | ND GROUPS | | | |
| HPLC-SEC | diameter ± SD (nm) | 6 ± 2 | 12 ± 2 | 16 ± 2 | >18 |

| | | MSP1D1:Li = 1:40 | | | |
|---|---|---|---|---|---|
| EM | weight (%) | 6 | 65 | 7 | 2 |
| | diameter ± SD (nm) | 9.6 ± 0.4 | 11.9 ± 1.0 | 15.0 ± 0.6 | 17.5 ± 0.9 |
| DLS | diameter ± SD (nm) | 11.2 ± 1.9, 100% | | | |
| SAXS | Rg (nm) | 5.2 ± 0.03 | | | |
| | | MSP1E3D1:Li = 1:80 | | | |
| EM | weight (%) | 2 | 78 | 17 | 2 |
| | diameter ± SD (nm) | 9.6 ± 0.3 | 11.9 ± 0.9 | 15.1 ± 0.8 | 19.2 ± 1.0 |
| DLS | diameter ± SD (nm) | 11.2 ± 1.9, 100% | | | |
| SAXS | Rg (nm) | 5.2 ± 0.03 | | | |
| | | MSP1E3D1:Li = 1:150 | | | |
| EM | weight (%) | 2 | 36 | 45 | 17 |
| | diameter ± SD (nm) | 9.4 ± 0.4 | 12.3 ± 1.1 | 15.8 ± 1.1 | 19.8 ± 1.5 |

| | | |
|---|---|---|
| DLS | diameter ± SD (nm) | 14.1 ± 3.2, 99.8% |
| SAXS | — | — |

Next, the inventors assembled and characterized ND with two different MSPs: MSP1D1 and MSP1E3D1 keeping the lipid composition constant at PS:GC=80:20% and changing the MSP to lipid ratio (Table 3). DLS measurements showed good agreement with EM data, as both ND populations assembled at MSP1D1 at 1:40 and MSP1E3D1 at 1:80 showed a main fraction with diameter of 11.2 nm which is very close to that determined by EM˜11.9 nm. SAXS measurements of these two groups also showed similar scattering curves and radii of gyration (Rg) ˜5.2 nm. The ND assembled with MSP1E3D1:lipids ratio at 1:150 were larger in size ˜14.1±3.2 nm and more heterogeneous.

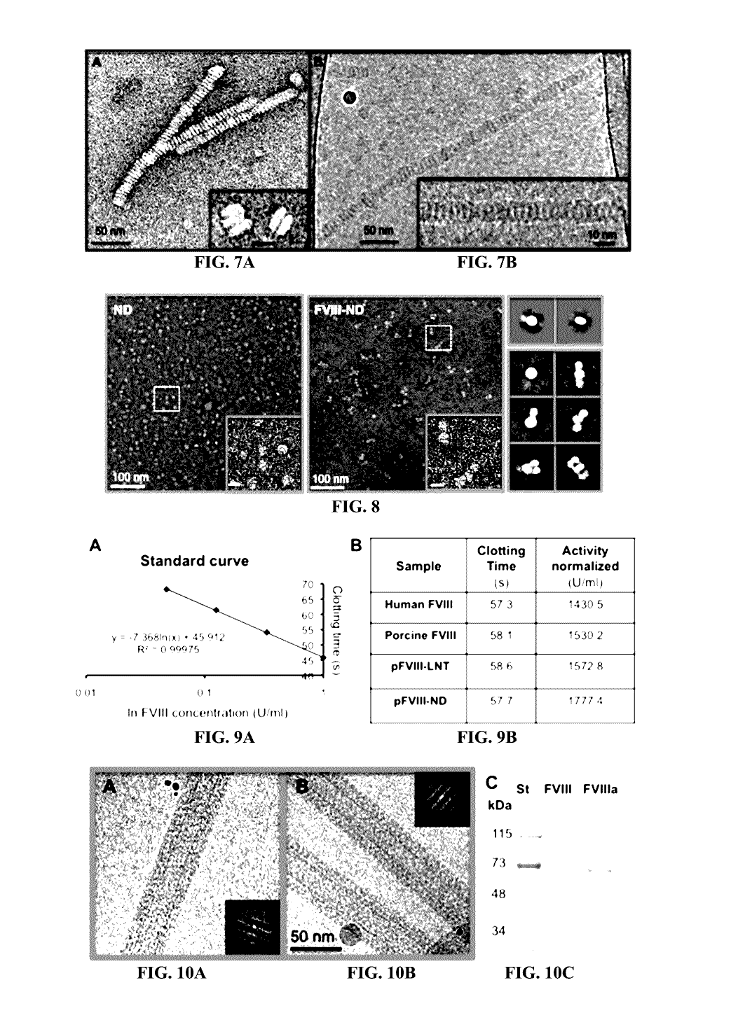

Stacking of PS-GC-ND in the presence of Ca²⁺. As Ca²⁺ ions are required for the function of the proposed in this study clotting proteins and complexes, examined their effect on the ND assembled in Table 1. No stacking was observed for the ND assembled in the absence of Ca²⁺ ions (Table 1). Adding 5 mM CaCl₂ to ND assembled at MSP1E3D1:lipids=1:80 and 1:150 ratios showed preferential stacking of the ND with larger diameter ˜15 nm. The stacking was more pronounced for the ND assembled at higher MSP1E3D1 to lipids ratio of 1:150 showing a significant increase in the stacks' length over 60 minutes. Increasing the Ca²⁺ concentration lead to a proportional increase in the length (FIGS. 7A and 7B). Above 10 mM Ca²⁺, the stacks start aggregating through lateral contact and Y branching (FIG. 7A). Cryo-EM of the ND stacks in holey carbon grids showed that the stacks can grow in micron lengths and are sufficiently well organized for further structure analysis (FIG. 7B). The stacking was found to be reversible upon adding 10 mM EDTA, as Ca²⁺ chelating agent. The selectivity of the stacking mechanism for nanodiscs with different size can be employed to obtain highly segregated ND populations with the same structure (size and shape) by alternating the stacking and unstacking mechanisms.

Completing the phase diagram for the GC-PS-ND. ND with three different types of scaffolding proteins can be assembled: MSP1D1, MSP1E3D1, and the ApoA1 (available from Sigma-Aldrich) and five different types of lipids: DOPS, DOPE, GC, DOPC Cholesterol (available form Avanti Polar Lipids) significant for the biological function of the proteins proposed in this study. The ND at different lipid compositions and MSP to lipids ratio, as detailed in Table 3 using the following lipids and proteins.

TABLE 3
Lipids and proteins.

| Lipids: | MSP: |
|---|---|
| (DOPS) 18:1 PS, | (Apo A-I) GLOB-H1-H2-H3-H4-H5-H6-H7- |
| C₄₂,H₇₇,NO₁₀PNa | H8-H9-H10 |
| 24:1 Galactosyl(β) | (MSP1D1) HisTev-H0.5-H2-H3-H4-H5-H6- |
| Ceramide C₄₈H₉₁NO₈. | H7-H8-H9-H10 |
| (DOPE) 18:1 (Δ9-Cis) PE, | (MSP1E3D1) HisTev-H0.5-H2-H3-H4-H5- |
| C₄₁H₇₈NO₈P. | H6-H4-H5-H6-H7-H8-H9-H10 |
| (DOPC) 18:1 (Δ9-Cis) PC, | |
| C₄₄H₈₄NO₈P | |
| (CHOLESTEROL), | |
| C₂₇H₄₆O, Mw = 386.355 | |

The size distribution of the ND are obtained by three biophysical techniques SEC, DLS and CD before imaging with negative stain EM, Cryo-EM and SAXS, as described hereinabove. The MSP to lipids ratio and lipid compositions can then be optimized as described hereinabove to achieve homogenous ND populations with CD spectra with higher alpha-helical content (˜20% higher) than the MSP alone, for MSP1D1, and DOPC-ND. A more stable and homogenous organization of the ND, due to a more structured “belt” holding the ND bilayer, can be provided.

Adding Cholesterol to the system will further the rigidity of the ND affects their size distribution. The cycle of alternating best MSP to lipid ratio for a given lipid composition can be repeated for the tertiary lipid mixtures proposed in Table 4. Several ND populations can also be selected and assessed by EM and chosen for further structure analysis by Cryo-EM.

It is also possible to stack ND in the presence of Ca²⁺ and Mg²⁺ ions that are biologically significant for FVIII and the serine proteases' function and assembly. The stacking can additionally separate the ND by size and provide information on the lipid-protein organization in the ND assembly by structural Cryo-EM.

TABLE 4
Parameters for ND lipid phase diagram studies.

| | MSP:Li | PS:GC:PE | PS:PC:PE | PS:GC:Chol | PS:PC:Chol |
|---|---|---|---|---|---|
| MSP | (%) | (%) | (%) | (%) | (%) |
| MSP1D1 | 1:40 | 100.0* | | | |
| | 1:50 | 80:20.0 | 80:20:0 | 75:20:5 | 80:15:5 |
| | as needed | 70:20:10 | 70:20:10 | 70:20:10 | 80:10:10 |
| MSP1E3D1 | 1:60 | 100.0* | | | |
| | 1:80 | 80:20.0 | 80:20.0 | **** | **** |
| | as needed | *** | *** | | |
| Apo A1 | ** | 100.0* | | | |
| | | 80:20.0 | 80:20.0 | **** | **** |
| | | *** | *** | | |

*The MSP to lipids ratio will be tested first with 100% DOPS.
** ApoA1 will be first tested for the best lipid compositions ad MSP1D1 to lipids ratio.
*** The tertiary mixtures will be tested at the optimal MSP to lipids ratio.
**** It has been shown that restricting the lipid bilayer by MSP has a similar effect as adding Cholesterol.

FVIII-ND complexes as therapeutics for the treatment of Hemophilia A and related blood disorders. As shown hereinabove, the FVIII-ND complexes have been used in clotting tests in vitro and in vivo. Purified FVIII variants are stable for more than two years, as monitored by the aPTT FVIII activity tests performed and conducted before each of the described biophysical and structural studies.

FVIII-ND complex assembly and characterization. Both recombinant human and porcine FVIII have been made on ND and characterized the FVIII macromolecular organization by negatively stained (NS-EM) (FIG. 8). The NS-EM studies of the ND alone and the FVIII-ND complexes clearly showed the macromolecular distribution of the protein when bound to the ND (FIG. 8). Reference free 2D classification of the ND, porcine FVIII and porcine FVIII-ND showed that the protein binds more often on both sides of the ND membrane than on a single side (FIG. 7). The inventors have further calculated 3D structures for the porcine FVIII-ND complexes by selecting only the one with FVIII bound to one side of the ND. After performing consecutive 2D reference free alignment to separate homogenous FVIII-ND complexes and applying single particle reconstruction algorithms several 3D reconstruction in the 25 Å resolution range can be achieved. The calculated porcine FVIII-ND 3D reconstructions show that a variability in the macromolecular organization of the FVIII molecules on the ND membrane surface exists that results in slightly different FVIII membrane-bound organization (FIG. 8). This variability in the FVIII membrane-bound organization was attributed to the flexibility of the C domains and the lack of close neighbors as observed for the FVIII 3D crystals in solution and the membrane-bound FVIII 2D and 3D crystals.

An added benefit of working with membrane-associated proteins is that they do not alter the ND's size and lipid phase diagram as dramatically as integral membrane proteins such as bacteriorhodopsin. NS-EM studies confirmed that binding of FVIII to ND do not affect their shape and geometry (FIGS. 7 and 8). Using the techniques outlined hereinabove, sufficient homogeneity can be achieved in the macromolecular organization of the membrane-associated proteins-ND complexes to calculate subnanometer structures by Cryo-EM at different lipid and protein environments.

Characterization and Assembly of the FVIII-ND Complexes. Porcine FVIII (pFVIII) can be purified and characterized. Human FVIII (hFVIII) variants can be obtained from the Gulf States Hemophilia and Thrombophilia center. All proteins can be characterized functionally with the aPTT (FIGS. 9A and 9B). The advantage of the ROTEM analysis using Thromboelastography is that it can be performed with whole blood, whereas the aPTT tests can be only performed with blood plasma, thus omitting the cellular components that are critical for the efficiency and potency of the clotting factors in vivo. The purity of all clotting proteins can be determined by SDS-polyacryl gel electrophoresis (PAGE). All functional assays can be performed as described hereinabove.

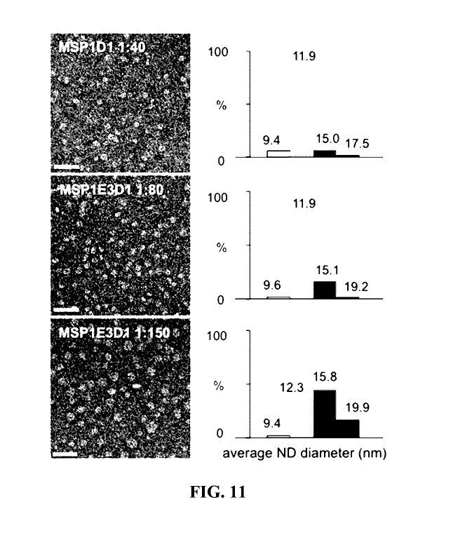

Therapeutic Effect of FVIII-ND In Vivo. Activity tests were carried out with the human and porcine FVIII in solution and when bound to ND and LNT didn't show a change in the FVIII function when measured against FVIII deficient plasma in vitro, as shown in FIGS. 9A-9B. Membrane binding improves FVIII biochemical and biophysical properties, therefore, the inventors determined the effect of ND for FVIII function in vivo and tested the lipid ND as a delivery system for FVIII. It was found that the porcine FVIII-ND complexes when injected in FVIII (FVIII-KO) deficient mice showed a pronounced pro-coagulant effect, which was stronger than that of the FVIII alone (FIGS. 10A to 10C). Therefore binding of porcine FVIII to ND prior to its injection in hemophilic mice significantly improves the therapeutic function of the protein. The stabilizing effect of the ND coupled with their pro-coagulant effect could have the added benefit of reducing the amount of FVIII required for preventing bleeding episodes in Hemophilia A patients and thus lower the cost of treatment.

FIG. 11 shows the size distribution of PS-GC-ND assembled from PS:GC=4:1 ratio and two MSP: MSP1D1 and MSP1E3D1 at different MSP to lipid ratio. Left. EM micrographs of negatively stained ND adsorbed on amorphous carbon. Scale bar 50 nm. Right. Size distribution graphs as measured from the NS-EM micrographs.

Cryo-EM and SPA reconstruction of the FVIII-ND complexes. To improve the FVIII-ND complexes for structure determination the homogeneity and monodispersity can be optimized by, e.g., introducing a second SEC step to separate the FVIII-ND complexes from single FVIII and ND. The 3D reconstruction of the FVIII-ND complexes can be determined from Cryo-EM data.

It is possible to activate FVIII when bound to ND with thrombin following known protocols and the membrane-bound FVIIIa can be further stabilized by adding active site inhibited FIXa. As such, the FVIIIa-ND and FVIIIa-FIXa structures can be refined following the methodology developed for the FVIII-ND complexes.

Biophysical characterization of the FVIII-ND complexes. Additional biophysical techniques, such as dynamic light scattering (DLS) and small angle X-ray scattering (SAXS), can also be used to evaluate the assembly of FVIII-ND and FVIIIa-FIXa-ND.

Optimization of the FVIII-ND complexes therapeutic effect in vivo. To further optimize the FVIII-ND construct already tested in FVIII deficient mice in vivo, a standardized thromboelsatography test can be used to evaluate the role of the cellular components for the coagulation testes previously carried out in vitro, such as the aPTT and chromogenix tests, which are performed against FVIII deficient plasma only. Purified recombinant porcine FVIII can be used, e.g., to test human FVIII deficient (hemophilia A) blood with or without presence of antibody and compare to the aPPT test and snip-tail tests kinetics (See FIG. 4A). Based on the functional data, site-specific mutations can be made to customize the therapeutic effect in vitro.

Develop LNT as an alternative membrane nano-platform to the ND for structural and functional studies of clotting proteins and complexes.

LNT can also be optimized at the same lipid composition as the ND and organize helically recombinant FVIII for high-resolution structure determination by Cryo-EM, following the methodology taught hereinabove.

Cryo-EM of helically organized FVIII bound to LNT. Both human and porcine FVIII on LNT have been organized helically and achieved a 3D reconstruction of the membrane-bound proteins on the LNT surface at 20 Å by Cryo-EM and helical analysis. The human and porcine FVIII showed consistently different helical organization when bound to LNT at 80% PS and 20% GC lipid composition. Both proteins however, formed stable dimers when bound to the PS-rich LNT surface (data not shown). To resolve the FVIII dimeric organization when bound to a PS rich membrane, the Cryo-EM structure of porcine FVIII bound to LNT can be further defined to 15 Å. By applying electron tomography (ET) and single particle tomography (SPT) reconstruction algorithms the inventors confirmed that the helically organized membrane-bound FVIII forms dimers on LNT and is not an artifact of the imposed helical symmetry. FVIII dimeric organization was not observed in the absence of PS-rich membrane, as published from NS-EM studies of FVIII-von Willebrandt (vWF) complexes in solution, and as shown herein using NS-EM and Cryo-ET, and SPT studies.

FVIII organization as dimers on the LNT surface and implications for the FVIIIa-FIXa complex assembly. Based on the helical reconstruction of membrane-bound porcine FVIII at 15 Å and the known crystal structure for the site inhibited FIXa and its Gla domain a dimeric model of the FVIIIa-FIXa complex membrane-bound organization has been developed in silico. The previously identified (biochemically and biophysically) residues at the FVIIIa-FIXa and FVIIIa-FIXa-membrane interfaces fitted in the proposed model further validating the proposed “supre-tenase” complex assembly (data not shown). The FVIII protein-protein interface within the membrane-bound dimer was mapped and compared to the differences in sequence between the human and porcine FVIII in this region. The amino acid residues at a radius <8.7 Å from the monomer:monomer interface were selected and compared for both human and porcine amino acid sequences. Mapping the residues at the FVIII monomer:monomer interface will help to understand the implications of the evolutionary aspect on the FVIII membrane-bound organization on LNT. There are 33 interactions between the FVIII monomer-monomer interface that are <8.7 Å apart. From these 33, 14 of these residues differ between the human and porcine FVIII sequence and 8 have known Hemophilia A missense mutations. Mutating the residues which differ between the human and porcine construct can affect the membrane-bound organization, whereas mutating the residues identified as causing mild to moderate HA from the HA mutations database. Thus, using the present invention it is possible to modulate the function of the FVIII-membrane constructs for therapeutic applications.

Cryo-EM of FVIII and activated FVIII (FVIIIa) helically organized on LNT. As described hereinabove, the inventors have collected high resolution Cryo-EM data of membrane-bound FVIII and activated FVIII (FVIIIa) helically organized on LNT at 1.4 Å/pixels. (FIGS. 10A to 10C). 3D reconstructions show that subnanometer resolution (˜8-10 Å) can be attained, as defined form the Fourier shell correlation (FSC) criterion (data not shown). At this resolution, modeling the protein-protein and protein-protein interactions can be used to identify FVIII co-factor activity. The data described herein, indicates that there is no significant domain re-organization upon thrombin activation of membrane-bound FVIII. This finding may be important to the geometry of the FVIIIa-FIXa complex assembly and can be used to optimize multiple FVIII constructs. The various constructs shown herein, e.g., on LNT and ND, are thus useful for therapeutic applications.

Cryo-EM and Helical Reconstruction of FVIII and FVIIIa Bound to LNT. Helically organized FVIII-LNT and FVIIIa-LNT can be made. Using the present invention, differential maps can be calculated to evaluate the macromolecular interaction interfaces important for the FVIIIa membrane-bound organization and activation on the LNT surface. The FVIII monomer-monomer interface will be further refined to create a structure-based model of FVIII activation by collecting more high-resolution data at a better signal-to-noise with a new direct electron (DE50) camera. Using the present invention, the significance of the membrane-bound FVIII dimeric organization for FVIII activation and function can be analyzed by flexible fitting of the known atomic structures and functional assays and modeling of membrane-bound FVIIIa-FIXa complexes as shown hereinabove. Protein-protein and protein lipid interfaces by molecular dynamics simulation can also be defined as described hereinabove.

Functional tests of FVIII and FVIIIa in solution and when bound to LNT. The quality and activity of the FVIII and FVIII-LNT samples will be tested by SDS-PAGE (FIGS. 10A to 10C) and FVIII activity tests as described hereinabove. Stabilizing the recombinant FVIII by binding to membrane nano-mimetics can have the added benefit of dramatically improving the stability of the protein and its potency, thus reducing the cost of Hemophilia A treatments, when compared to current costs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Fay P J. Activation of factor VIII and mechanisms of cofactor action. Blood Rev 2004; 18: 1-15.
2. Fay P J. Factor VIII structure and function. Intern J Hematol 2006; 83: 103-108.
3. Pittman D D, Kaufman R J. Proteolytic requirements for thrombin activation of anti-haemophilic factor (factor VIII). Proc Natl Acad Sci USA 1988; 85: 2429-2433.
4. Fay P J, Smudzin T M. Characterisation of the interaction between the A2 subunit and A1/A3-C1-C2 dimer in human factor VIIIa. J Biol Chem 1992; 267: 13246-13250.
5. Wakabayashi H, Fay P J. Identification of residues contributing to A2 domain dependent structural stability in factor VIII and factor VIIIa. J Biol Chem 2008; 283: 11645-11651.
6. Shen B W, Spiegel P C, Chang C H, et al. The tertiary structure and domain organisation of coagulation factor VIII. Blood 2008; 111: 1240-1247.
7. Derrick T S, Kashi R S, Durrani M, et al. Effect of metal cations on the conformation and inactivation of recombinant human factor VIII. J Pharm Sci 2004; 93: 2549-2557.
8. Lollar P, Parker C G. Subunit structure of thrombin-activated porcine factor VIII. Biochemistry 1989; 28: 666-674.
9. Fay P J, Haidaris P J, Smudzin T M. Human factor VIIIa subunit structure. Reconstruction of factor VIIIa from the isolated A1/A3-C1-C2 dimer and A2 subunit. J Biol Chem 1991; 266: 8957-8962.
10. Duffy E J, Parker E T, Mutucumarana V P, et al. Binding of factor VIIIa and factor VIII to factor IXa on phospholipid vesicles. J Biol Chem 1992; 267: 17006-17011.
11. Pratt K P, Shen B W, Takeshima K, et al. Structure of the C2 domain of human factor VIII at 1.5 Å resolution. Nature 1999; 402: 439-442.
12. Lenting P J, van Mourik J A, Mertens K. The life cycle of coagulation factor VIII in view of its structure and function. Blood 1998; 92: 3983-3996.
13. Hsu T C, Pratt K P, Thompson A R. The factor VIII C1 domain contributes to platelet binding. Blood 2008; 111: 200-208.
14. Ahmad S S, Walsh P N. Coordinate binding studies of the substrate (factor X) with the cofactor (factor VIII) in the assembly of the factor X activating complex on the activated platelet surface. Biochemistry 2002; 41: 11269-11276.
15. Furie B, Furie B C. Mechanisms of thrombus formation. N Engl J Med 2008; 359: 938-949.
16. Pipe S W. Haemophilia: new protein therapeutics. Hematology Am Soc Hematol Educ Program 2010; 2010: 203-209.
17. Shaw A W, Pureza V S, Sligar S G, et al. The local phospholipid environment modulates the activation of blood clotting. J Biol Chem 2007; 282: 6556-6563.
18. Ruprecht J, Nield J. Determining the structure of biological macromolecules by transmission electron microscopy, single particle analysis and 3D reconstruction. Progr Biophys Mol Biol 2001; 75: 121-164.
19. Grushin K, Miller J, Dalm D, et al. Lack of recombinant factor VIII B-domain induces phospholipid vesicle aggregation: implications for the immunogenicity of factor VIII. Haemophilia 2014; 20: 723-731.
20. Henderson R. The potential and limitations of neutrons, electrons and X-rays for atomic resolution microscopy of unstained biological molecules. Quart Rev Biophys 1995; 28: 171-193.
21. Frederik P M, Hubert D H W. Cryoelectron microscopy of liposomes. Methods Enzymol 2005; 391: 431-448.
22. Katayama H, Wang J, Tama F, et al. Three-dimensional structure of the anthrax toxin pore inserted into lipid nanodiscs and lipid vesicles. Proc Natl Acad Sci USA 2010; 107: 3453-3457.
23. Akkaladevi N, Hinton-Chollet L, Katayama H, et al. Assembly of anthrax toxin pore: lethal-factor complexes into lipid nanodiscs. Protein Sci 2013; 22: 492-501.
24. Gatti L, Mannucci P M. Use of porcine factor VIII in the management of seventeen patients with factor VIII antibodies. Thromb Haemost 1984; 51: 379-384.
25. Hay C R, Lozier J N, Lee C A, et al. Safety profile of porcine factor VIII and its use as hospital and home-therapy for patients with haaemophilia-A and inhibitors: the results of an international survey. Thromb Haemost 1996; 75: 25-29.
26. Lollar P, Parker E T. Structural basis for the decreased procoagulant activity of human factor VIII compared to the porcine homolog. J Biol Chem 1991; 266: 12481-12486.
27. Lollar P, Parker E T, Fay P J. Coagulant properties of hybrid human/porcine factor VIII molecules. J Biol Chem 1992; 267: 23652-23657.
28. Doering C B, Healey J F, Parker E T, et al. High level expression of recombinant porcine coagulation factor VIII. J Biol Chem 2002; 277: 38345-38349.
29. Miller J, Dalm D, Koyfman A Y, et al. Helical organisation of blood coagulation factor VIII on lipid nanotubes. J Vis Exp 2014; Epub ahead of print.
30. Stoilova-McPhie S, Grushin K, Dalm D, et al. Lipid nanotechnologies for structural studies of membrane-associated proteins. Proteins 2014; 82: 2902-2909.
31. Ritchie T K, Grinkova Y V, Bayburt T H, et al. Chapter 11—Reconstitution of membrane proteins in phospholipid bilayer nanodiscs. Methods Enzymol 2009; 464: 211-231.
32. Pace C N, Vajdos F, Fee L, et al. How to measure and predict the molar absorption coefficient of a protein. Protein Sci 1995; 4: 2411-2423.
33. Laemmli U K. Cleavage of structural proteins during the assembly of the head of bacteriophage T4. Nature 1970; 227: 680-685.
34. Tang G, Peng L, Baldwin P R, et al. EMAN2: an extensible image processing suite for electron microscopy. J Struct Biol 2007; 157: 38-46.
35. Parmenter C D J, Stoilova-McPhie S. Binding of recombinant human coagulation factor VIII to lipid nanotubes. FEBS Lett 2008; 582: 1657-1660.
36. Blanchette C D, Law R, Benner W H, et al. Quantifying size distributions of nanolipoprotein particles with single-particle analysis and molecular dynamic simulations. J Lipid Res 2008; 49: 1420-1430.
37. Denisov I G, Grinkova Y V, Lazarides aa, et al. Directed self-assembly of monodisperse phospholipid bilayer Nanodiscs with controlled size. J Am Chem Soc 2004; 126: 3477-3487.
38. Stoilova-McPhie S, Lynch G C, Ludtke S, et al. Domain organisation of membrane-bound factor VIII. Biopolymers 2013; 99: 448-459.
39. Morrissey J H, Pureza V, Davis-Harrison R L, et al. Blood clotting reactions on nanoscale phospholipid bilayers. Thromb Res 2008; 122 (Suppl): S23-S26.
40. Grinkova Y V, Denisov I G, Sligar S G. Engineering extended membrane scaffold proteins for self-assembly of soluble nanoscale lipid bilayers. Protein engineering design selection PEDS 2010; 23: 843-848.
41. Biro E, Akkerman J W N, Hoek F J, et al. The phospholipid composition and cholesterol content of platelet-derived microparticles: a comparison with platelet membrane fractions. J Thromb Haemost 2005; 3: 2754-2763.
42. Demel Ra, Paltauf F, Hauser H. Monolayer characteristics and thermal behavior of natural and synthetic phosphatidylserines. Biochemistry 1987; 26: 8659-8665.
43. Smaby J M, Kulkarni V S, Momsen M, et al. The interfacial elastic packing interactions of galactosylceramides, sphingomyelins, and phosphatidylcholines. Biophys J 1996; 70: 868-877.
44. Stoylova S S, Lenting P J, Kemball-Cook G, et al. Electron crystallography of human blood coagulation factor VIII bound to phospholipid monolayers. J Biol Chem 1999; 274: 36573-36578.
45. Stoilova-McPhie S, Villoutreix B O, Mertens K, et al. 3-Dimensional structure of membrane-bound coagulation factor VIII: modeling of the factor VIII heterodimer within a 3-dimensional density map derived by electron crystallography. Blood 2002; 99: 1215-1223.
46. Stoylova S, Mann K G, Brisson A. Structure of membrane-bound human factor Va. FEBS Lett 1994; 351: 330-334.
47. S, Gray E, Barrowcliffe T W, et al. Structural determination of lipid-bound human blood coagulation factor IX. Biochim Biophys Acta 1998; 1383:175-178.

What is claimed is:

1. A lipid nanodisk or nanotube composition comprising:
a lipid composition comprising phosphatidylserine and galactosylceramide and a membrane-bound Factor VIII protein, a membrane-bound Factor IX protein, a membrane-bound Factor VIII-Factor IX protein complex, or a membrane-bound Factor V and Factor X proteins and their complex in or about the lipid nanodisks or nanotubes.

2. The composition of claim 1, wherein the membrane-bound Factor VIII is a full length or B-domain deleted variant of recombinant FVIII.

3. The composition of claim 1, wherein the phosphatidylserine and galactosylceramide lipids and any derived lipids are at a ratio from 20 to 80 percent by volume.

4. The composition of claim 1, wherein the phosphatidylserine is used as a 20 to 50%, or 50 to 80% liquid composition.

5. The composition of claim 1, wherein the membrane-bound Factor VIII to lipid ratio is from 1:47 to 1:72.

6. The composition of claim 1, wherein the membrane-bound Factor VIII to lipid ratio is from 1:40 to 1:150.

7. The composition of claim 1, wherein the nanodisk is in a phosphatidylserine 80% lipid composition and 1:47 membrane-bound Factor VIII to lipid ratio.

8. The composition of claim 1, wherein the composition further comprises one or more amphiphilic membrane scaffolding proteins (MSP) and/or their derivatives of Apolipoproteins I.

9. The composition of claim 1, wherein the Factor VIII protein, the Factor IX protein, the Factor VIII-Factor IX protein complex, or the Factor V, Factor X proteins and their complex is an activated form of the protein.

10. The composition of claim 1, wherein the Factor VIII is activated truncated protein that forms a functional intrinsic tenase complex with a human FIXa on a negatively charged phospholipid surface.

11. A method of making a lipid nanodisk or nanotube comprising:
dissolving phosphatidylserine and galactosylceramide lipids in an organic solvent;
evaporating the organic solvent under a noble gas;
reconstituting the phosphatidylserine and galactosylceramide in an aqueous buffered solution with Na cholate;
warming and sonicating the phosphatidylserine and galactosylceramide until they are in solution;
adding a membrane-bound Factor VIII protein, a membrane-bound Factor IX protein, a membrane-bound Factor VIII-Factor IX protein complex, or a membrane-bound Factor V-Factor X protein complex into the phosphatidylserine and galactosylceramide; and
removing the Na Cholate with beads, wherein the NaCholate and beads are removed by centrifugation.

12. The method of claim 11, wherein the membrane-bound Factor VIII is full-length of a B-domain deleted variant of recombinant FVIII.

13. The method of claim 11, the phosphatidylserine and galactosylceramide lipids and any derived lipids are at a ratio from 20 to 80 percent by volume.

14. The method of claim 11, wherein the phosphatidylserine is used as a 20 to 50%, or 50 to 80% liquid composition.

15. The method of claim 11, wherein the membrane-bound Factor VIII to lipid ratio is from 1:47 to 1:72.

16. The method of claim 11, wherein the membrane-bound Factor VIII to lipid ratio is from 1:40 to 1:150.

17. The method of claim 11, wherein the nanodisk is in a phosphatidylserine 80% lipid composition and 1:47 membrane-bound Factor VIII to lipid ratio.

18. The method of claim 11, wherein the composition further comprises one or more amphiphilic membrane scaffolding proteins (MSP) and/or their derivatives of Apolipoproteins I.

19. The method of claim 11, wherein the Factor VIII protein, the Factor IX protein, the Factor VIII-Factor IX protein complex, or the Factor V and Factor X proteins complex is an activated form of the protein.

20. The method of claim 11, wherein the Factor VIII is activated truncated protein that forms functional an intrinsic tenase complex with a human FIXa on a negatively charged phospholipid surface.

21. A method of treating a disease of blood coagulation comprising:
identifying a subject in need of treatment for the disease of blood coagulation caused by a mutation in at least one of Factor V, Factor VIII, Factor IX, or Factor X; and
providing the subject with a therapeutically effective amount of a phosphatidylserine and galactosylceramide lipids nanodisk or nanotube composition comprising a membrane-bound Factor VIII protein, a membrane-bound Factor IX protein, a membrane-bound Factor VIII-Factor IX protein complex, or a membrane-bound Factor V and Factor X proteins complex, wherein the Factor V, Factor VIII, Factor IX, or Factor X are provided in an active or inactive form.

22. A method of determining the effectiveness of a candidate drug that impacts Factor V, VIII, IX and/or X activity, the method comprising:
(a) obtaining a serum or plasma from a normal subject and a subject with an abnormality in blood clotting;
(b) preparing a stable lipid nanodisk or nanotube comprising phosphatidylserine and galactosylceramide that comprises at least one of: a membrane-bound Factor VIII protein, a membrane-bound Factor IX protein, a membrane-bound Factor VIII-Factor IX protein complex, or a membrane-bound Factor V-Factor X protein complex;
(c) combining the serum or plasma from the normal and from the abnormal subjects; and
(d) imaging the membrane-bound Factor VIII protein, the membrane-bound Factor IX protein, the membrane-bound FactorVIII-Factor IX protein, or the membrane-bound Factor VIII-Factor IX protein complex in the normal and the abnormal serum by at least one of electron microscopy or single-particle analysis;
(e) adding the candidate drug to the nanodisks or nanotubes, and
(f) imaging the nanodisks or nanotubes in the normal and the abnormal serum by at least one of electron microscopy or single-particle analysis to determine the structural differences in nanodisks or nanotubes comprising the membrane-bound Factor VIII protein, the membrane-bound Factor IX protein, the membrane-bound FactorVIII-Factor IX protein, or the membrane-bound Factor VIII-Factor IX protein complex in the presence or absence of the candidate drug.
